(19) United States
(12) Patent Application Publication
(10) Pub. No.: US 2020/0135300 A1
(43) Pub. Date: Apr. 30, 2020

(54) APPLYING LOW COVERAGE WHOLE GENOME SEQUENCING FOR INTELLIGENT GENOMIC ROUTING

(71) Applicant: Color Genomics, Inc., Burlingame, CA (US)

(72) Inventors: Gilad Mishne, Albany, CA (US); Jeremiah Ginsberg, Park City, UT (US); Lawrence Sean Hon, Burlingame, CA (US); William Michael Stedden, Emeryville, CA (US); Julian Homburger, San Francisco, CA (US)

(21) Appl. No.: 16/665,508

(22) Filed: Oct. 28, 2019

Related U.S. Application Data
(60) Provisional application No. 62/751,233, filed on Oct. 26, 2018.

(51) Int. Cl. G16B 40/20; C12Q 1/6869; G16B 40/10; G16B 20/00; G16B 5/00; G06N 7/02; G06F 17/18; G16B 30/10; G16B 30/20

(52) U.S. Cl. CPC G16B 40/20; C12Q 1/6869; G16B 40/10; G16B 20/00; G16B 30/20; G06N 7/023; G06F 17/18; G16B 30/10; G16B 5/00

(57) ABSTRACT

Techniques for next generation sequencing (NGS), and more particularly, to techniques for applying low coverage whole genome sequencing (lcWGS) in genome wide association studies (GWAS). One aspect includes performing a lcWGS of a biological sample from a subject to obtain a set of reads, determining an inference of a phenotype from the set of reads, obtaining self-reported data from the subject, executing a first query on eligibility criteria for a plurality of genomic routes to obtain a set of genomic routes that satisfy the first query, executing a second query on routing criteria for the set of genomic routes to obtain a subset of ranked genomic routes that satisfy the second query, and selecting one or more genomic routes from the subset of ranked genomic routes based on the ranking of each of the one or more genomic routes.

### CROSS-REFERENCE TO RELATED APPLICATION

The present application claims the benefit of priority to U.S. Provisional Application No. 62/751,233, filed Oct. 26, 2018, the entire contents of which are incorporated by reference herein for all purposes.

### FIELD OF THE INVENTION

The present disclosure relates generally to next generation sequencing (NGS), and more particularly, to techniques for applying low coverage whole genome sequencing (lcWGS) in genome wide association studies (GWAS) for intelligent genomic routing.

### BACKGROUND

Any two human genomes differ in millions of different ways. There are small variations in the individual nucleotides of the genomes such as single-nucleotide polymorphisms (SNPs) as well as many larger variations, such as deletions, insertions and copy number variations. Any of these may cause alterations in an individual's traits, or phenotype, which can be anything from disease risk to physical properties such as height. Prior to the introduction of genome wide association studies (GWAS), the primary method of investigation for variations was through inheritance studies of genetic linkage in families. This approach had proven highly useful towards single gene disorders. However, for common and complex diseases the results of genetic linkage studies proved hard to reproduce. A suggested alternative to linkage studies was a genetic association study. This study type asks if the allele of a genetic variant is found more often than expected in individuals with the phenotype of interest (e.g. with the disease being studied). Early calculations on statistical power indicated that this approach could be better than linkage studies at detecting weak genetic effects.

This framework for genetic association studies and the advent of biobanks and increased computing power enabled the association studies to expand to genome wide sequencing. A GWAS is an observational study of a genome-wide set of genetic variants in different individuals to see if any variant is associated with a trait. GWASs typically focus on associations between SNPs and traits like major human diseases, but can equally be applied to any other genetic variants and any other organisms. The primary output of GWASs are estimations of the relation between variants in particular loci of the genome and an observable trait or traits, such as height or prevalence of certain diseases. These estimations can in turn be used to derive insights as to the underlying biological pathways of the trait, understand gene function, predict genetic risk for disease in an individual given her genotype, and more.

The most common approach of GWASs is the case-control setup, which compares two large groups of individuals, one healthy control group and one case group affected by a disease. All individuals in each group are genotyped for the majority of common known SNPs. The exact number of SNPs depends on the genotyping technology, but are typically a few hundred thousand or more. For each of these SNPs it is then investigated if the allele frequency is significantly altered between the case and the control group. In such setups, the fundamental unit for reporting effect sizes is the odds ratio. The odds ratio is the ratio of two odds, which in the context of GWASs are the odds of disease for individuals having a specific allele and the odds of disease for individuals who do not have that same allele. When the allele frequency in the case group is much higher than in the control group, the odds ratio is higher than one, and vice versa for lower allele frequency. Additionally, a P-value for the significance of the odds ratio may be calculated using, for example, a chi-squared test. Finding odds ratios that are significantly different from one is the objective of the GWAS because this shows that a variant such as a SNP is associated with disease.

The cost of a Whole-Genome Sequencing (WGS) has decreased tremendously in recent years due to advances in next-generation sequencing technologies. Nevertheless, the cost of carrying out large-scale cohort studies using WGAS is still daunting. Past simulation studies with low-coverage WGS (1× to 10×) and ultra-low coverage WGS (coverage below 1×) have shown promise for using low coverage whole genome sequencing (lcWGS) in studies focused on variant discovery, association study replications, population genomics characterization, and more. Coverage (or depth) in nucleic acid sequencing is the number of unique reads that include a given nucleotide in the reconstructed sequence. Low coverage sequencing refers to the general concept of aiming for a low number of unique reads of each region of a sequence. By sampling across the whole genome at a low depth combined with imputation, it is possible to reliably detect and predict common variants in samples. IcWGS combined with imputation has been demonstrated to accurately assess common genetic variation, is as fast and affordable as a genotyping array and achieves similar technical accuracy, while still being able to capture new and common variants across diverse populations as with deeper coverage GWAS.

### BRIEF SUMMARY

Techniques are provided (e.g., a method, a system, non-transitory computer-readable medium storing code or instructions executable by one or more processors) for implementing lcWGS in GWAS.

In various embodiments, a method is provided that comprises: sequencing, at a processing system, a large set of samples with associated phenotypes using a low coverage sequencing with a focus on less common variants; collecting, by the processing system, for each locus in a genome, all samples from the large set of samples that have some observation at the respective locus; performing, by the processing system, statistical association for each locus of the genome having an observation using only the samples collected that have the observation; and combining, by the processing system, the statistical association performed for each locus using standard approaches for combining summary information from different GWAS performed on different populations.

In some embodiments, the large set of samples is greater than 100 samples, greater than 500 samples, or greater than 1000 samples. In some embodiments, each locus is a different fixed position on the genome. In some embodiments, the observation is a variant of similar sequences located at the respective locus, and where the variant is a single nucleotide polymorphism (SNP) or the variant is a large structural variant such as a microdeletion or aneuploidy. In some embodiments, the less common variants have a Minor Allele Frequency (MAF) of <1%.

In various embodiments, a method is provided that comprises: given a sample S requiring imputation, identifying, at a processing system, K most similar samples to S in a dataset of samples that have full genotyping information; building, by the processing system, an imputation reference panel using the K full-genome samples selected by aggregating their genotypes; and applying, by the processing system, the imputation reference panel in an imputation flow.

In some embodiments, the dataset is obtained from a database such as the 1000 genomes project, the HapMap Consortium database, or a proprietary database. In some embodiments, similarity between samples is computed using Identity-by-Descent estimation. In some embodiments, similarity between samples is computed using principal component analysis. In some embodiments, the imputation flow includes identifying stretches of shared haplotype in the K full-genome samples and missing genotypes for each patient sample can be filled in by copying alleles observed in matching reference haplotypes. In some embodiments, the imputation flow includes estimate missing haplotypes based on a simple heuristic or on an E-M algorithm or on more sophisticated coalescent models.

In various embodiments, a method is provided that comprises: sequencing, at a processing system, a sample to obtain a set of reads; identifying, by the processing system, within the reads one or more off-target reads; performing, by the processing system, statistical association for loci of the one or more off-target reads having an observation; and determining, by the processing system, an inference of a phenotype from the one or more off-target reads based on the statistical association.

In some embodiments, the inference include identifying a genetic ancestry of the sample, and consequently handling the sample differently in downstream processing. In some embodiments, the sequencing includes reducing or increasing targeted overall coverage to achieve a minimum total number of the off-targeted reads. In some embodiments, the method further comprises reducing or increasing targeted overall coverage in a subsequent low coverage whole genome sequencing assay to achieve a minimum total number of the off-targeted reads. In some embodiments, the sample is sequenced using low coverage sequencing.

In various embodiments, a method is provided that comprises: performing a first sequencing, at a processing system, of a sample to obtain a first set of reads; evaluating, at a processing system, coverage of the first set of reads; performing a second sequencing, at a processing system, of a sample to obtain a second set of reads based at least on the evaluation; performing, by the processing system, statistical association for loci of the first set of reads and/or the second set of reads having an observation; and determining, by the processing system, an inference of a phenotype from the first set of reads and the second set of reads based on the statistical association.

In some embodiments, the first sequencing is a high coverage whole genome sequencing and the second sequencing is a low coverage sequencing.

In some embodiments, the first sequencing is a low coverage whole genome sequencing and the second sequencing is a high coverage sequencing.

In various embodiments, a method is provided comprising: performing, at a processing system, a low coverage whole genome sequencing of a biological sample from a subject to obtain a set of reads; evaluating, at a processing system, coverage of the set of reads; performing, by the processing system, statistical association for loci of the set of reads having an observation; determining, by the processing system, an inference of a phenotype from the set of reads based on the statistical association; obtaining, by the processing system, self-reported data from the subject; executing, by the processing system, a first query on eligibility criteria for a plurality of genomic routes to obtain a set of genomic routes that satisfy the first query, where the first query includes the phenotype; executing, by the processing system, a second query on routing criteria for the set of genomic routes to obtain a subset of ranked genomic routes that satisfy the second query, where the second query includes the phenotype and at least one piece of information from the self-reported data; and selecting, by the processing system, one or more genomic routes from the subset of ranked genomic routes based on the ranking of each of the one or more genomic routes.

In some embodiments, the method further comprises providing, by the processing system, insight and/or information supporting a product, service, event, or benefit associated with each of the selected one or more genomic routes to the subject.

In some embodiments, the first query is executed on a query dependent model configured to obtain each genomic route of the plurality of genomic routes that satisfies the first query, and the second query is executed on a learning to rank model trained to rank or prioritize each genomic route of the set of genomic routes that satisfies the query.

In some embodiments, the method further comprises: identifying, by the processing system, within the set of reads one or more off-target reads; performing, by the processing system, a statistical association for loci of the one or more off-target reads having an observation; and determining, by the processing system, an inference of another phenotype from the one or more off-target reads based on the statistical association, where the second query includes the phenotype, the another phenotype, and at least one piece of information from the self-reported data.

In some embodiments, the method further comprises: performing, at the processing system, a high coverage whole genome sequencing of the sample to obtain another set of reads; and performing, by the processing system, statistical association for loci of the another set of reads having an observation, where the inference of the phenotype is determined from the set of reads and the another set of reads based on the statistical association of both the set of reads and the another set of reads.

In some embodiments, the insight and/or the information are provided to the subject without the phenotype. In other embodiments, the insight and/or the information are provided to the subject with the phenotype.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims

### BRIEF DESCRIPTION OF THE DRAWINGS

FIG. 1 shows a simplified block diagram of a process for low coverage whole genome sequencing in accordance with various embodiments.

FIG. 2 depicts a flowchart illustrating a process for low coverage genome wide association studies on rare bands in accordance with various embodiments.

FIG. 3 depicts a flowchart illustrating a process for imputation for low coverage sequencing in accordance with various embodiments.

FIG. 4 depicts an illustrative architecture of a computing system in accordance with various embodiments.

FIG. 5 depicts a flowchart illustrating a process for preparing or pre-processing genomic data in accordance with various embodiments.

FIG. 6 depicts a flowchart illustrating a process for generating one or more genomic routes in accordance with various embodiments.

FIG. 7 depicts a flowchart illustrating a process for querying, ranking, and selecting one or more genomic routes for a subject in accordance with various embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

As used herein and in the appended claims, the singular forms “a”, “an”, and “the” include plural referents unless the context clearly dictates otherwise. Thus, for example, reference to “a device” includes a plurality of such devices known to those skilled in the art, and so forth.

The term “nucleic acid,” as used herein, generally refers to a polymeric form of nucleotides of any length, either ribonucleotides, deoxyribonucleotides or peptide nucleic acids (PNAs) that comprise purine and pyrimidine bases, or other natural, chemically or biochemically modified, non-natural, or derivatized nucleotide bases. A nucleic acid can refer to a polynucleotide. The backbone of the polynucleotide can comprise sugars and phosphate groups, as can be found in ribonucleic acid (RNA) or deoxyribonucleic acid (DNA), or modified or substituted sugar or phosphate groups. A polynucleotide can comprise modified nucleotides, such as methylated nucleotides and nucleotide analogs. The sequence of nucleotides can be interrupted by non-nucleotide components. Thus the terms nucleoside, nucleotide, deoxynucleoside and deoxynucleotide can generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. These analogs can be derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired. The nucleic acid molecule can be a DNA molecule. The nucleic acid molecule can be an RNA molecule.

The terms “variant” and “derivative,” as used herein in the context of a nucleic acid molecule, generally refer to a nucleic acid molecule comprising a polymorphism. Such terms can also refer to a nucleic acid product that is produced from one or more assays conducted on the nucleic acid molecule. For example, a fragmented nucleic acid molecule, hybridized nucleic acid molecule (e.g., capture probe hybridized nucleic acid molecule, bead bound nucleic acid molecule), amplified nucleic acid molecule, isolated nucleic acid molecule, eluted nucleic acid molecule, and enriched nucleic acid molecule are variants or derivatives of the nucleic acid molecule.

Where a range of values is provided, it is understood that each intervening value between the upper and lower limits of that range, to the tenth of the unit of the lower limit, unless the context clearly dictates otherwise, is also specifically disclosed. Each smaller range between any stated value or intervening value in a stated range, and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention

### Low Coverage Whole Genome Sequencing

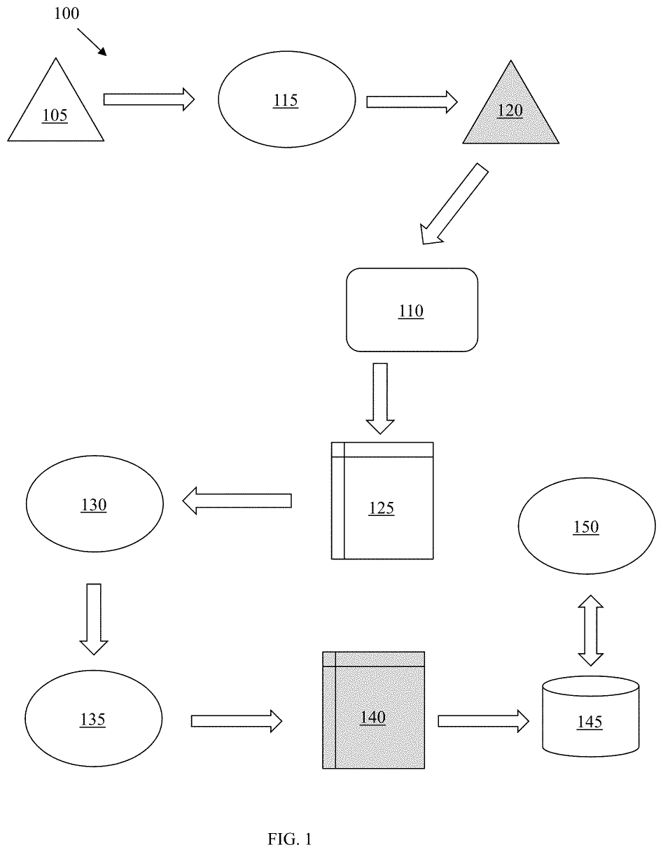

A typical sequencing-based genotyping technique uses “high coverage” sequencing (e.g., 30× average coverage), meaning that each base of the genome is covered on average by 30 sequencing reads. Such a technique requires considerable time and effort. The cost per sample for sequencing-based genotyping can be lowered by using “low-coverage” genome sequencing; e.g., about 1× to about 5× average coverage, or “ultra-low coverage”, e.g. coverage lower than 1×; e.g., about 0.01× to about 1×, and in certain embodiments about 0.5×. FIG. 1 is a block diagram that illustrates an example process 100 for low coverage whole genome sequencing according to some embodiments. The nucleic acids 105 in a plurality of samples may be prepared for a sequencer 110 in a wide variety of ways known in the art, often by de-naturing to release the nucleic acids, fragmentation to allow the short reads to begin sequencing from anywhere within the nucleic acid having the reference sequence, to hybridization or replication or amplification or size selection, among others, or some combination, which collectively are referenced herein as sample preparation 115. The resulting short nucleic acids 120 are then sequenced at low coverage by the sequencing process in sequencer 110 (e.g., about 0.01× to about 5× average coverage). The reads are recorded in an initial data structure 125 with a field holding data that represents each read sequence, e.g., single end, paired end, and mate pair sequencing data.

Prior to assembly, the quality of the sequencing data in the data structure 125, overall GC content, repeat abundance or the proportion of duplicated reads may be assessed in preassembly 130. For example, trimming low-quality data and reads resulting from PCR duplications can be performed with a variety of different software and scripts. Stand-alone error correcting, using a k-mer count approach can also be a useful alternative for many datasets. Failure to remove such abundant contaminant sequences can disrupt the assembly process (due to the high read depth compared with the nuclear genome) and may result in the production of chimeric and contaminated contigs. Once preassembly is complete, the trimmed and error corrected sequencing data in the data structure 125 are assembled in a genome assembly process 135, e.g., a de novo assembly process to generate genome sequence data stored in data structure 140, which is stored in a database 145. Once an assembly has been successfully performed, a quality control process 150 may be implemented to assess quality of the assembled genome sequence data or compare several assemblies using different methods. To harness the full potential of the genome sequence data, the genome sequence data in the data structure 140 may be annotated with biologically relevant information that can range from gene models to functional information, such as associated phenotypes.

### Low Coverage Genome Wide Association Studies on Rare Bands

To perform a GWAS, a dataset of genotype-phenotype pairs for a large number of individuals is needed. A genotype is the individual's genome, or a subset of it; a phenotype is the set of observations for which prediction is desired, e.g. height, disease status, and so on. Due to cost limitations, only a small subset of the individual's genome is typically mapped; often, a set of 500,000-2,000,000 loci are genotyped, out of approximately 3 billion total loci. These loci are selected so that the rest of the genome can be estimated, or imputed, with good accuracy, using known relations between sites in the human genome. However, imputation accuracy drops for variants that are less common across populations, so GWAS experiments are usually performed for high-frequency variants only. Frequency depends on population and observability, and often a cutoff such as “appears in 1% or more of a population” is applied, leaving 10-30 million loci for which a typical GWAS produce statistics. One limitation of this approach is that less frequent loci in the genome, but that may still have a strong statistical association with a trait, are not analyzed. In fact, it is well-accepted that the loci uncovered in current GWAS experiments are likely not causative for the traits they are associated with, but are rather highly correlated with other variants in the genome, which do drive the trait.

To overcome the challenges of analyzing less frequent loci in the genome (“rare or uncommon bands”) while maintaining low costs using low-coverage WGS, various embodiments are directed to a process that includes collecting a large set of samples with associated phenotypes, and sequence them using the low-coverage assay with a focus on less common variants (Minor Allele Frequency, or MAF<1%). In each sample, some of the genome will be sequenced at low coverage or depth, and the rest would have no genotype at all. In typical lcWGS coverage such as 1×, more than half of the genome will be genotyped in any given individual. The process may further include for each locus or site in the genome, collecting all samples that have some observation at the locus or site. This may be a small or large subset of all samples, or may even be all of them, depending on coverage in the particular set of samples. In typical IcWGS coverage such as 1X, the set of samples where a particular locus or site was sequenced will be a large subset of the total number of samples. Thereafter, perform statistical association for the locus or site using only the samples covering the locus or site. Once statistical association is performed separately for each locus or site, combine the statistics obtained for each locus or site using standard approaches for combining summary information from different GWAS performed on different populations, that is, correct for population structure and other confounding variants.

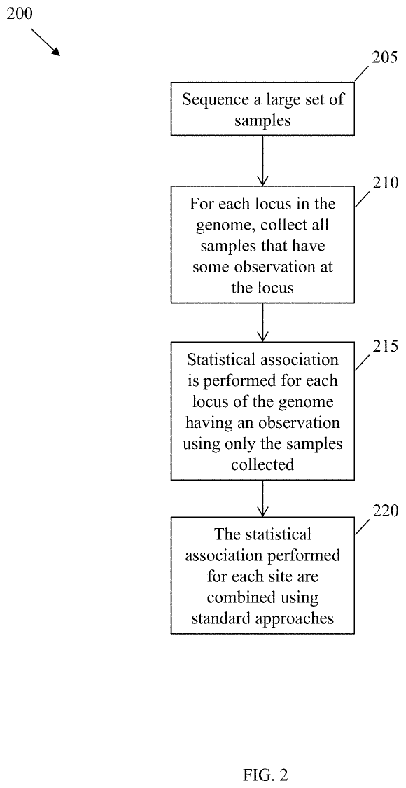

FIG. 2 is a block diagram that illustrates an example process 200 for a low-coverage assay with a focus on less common variants (MAF<1%), according to some embodiments. In step 205, a large set of samples with associated phenotypes are sequenced, for example as described with respect to FIG. 1. In some embodiments, the large set of samples is greater than 100 samples. In other embodiments, the large set of samples is greater than 500 samples. In yet other embodiments, the large set of samples is greater than 1000 samples. At step 210, for each locus in the genome, collect all samples from the large set of samples that have some observation at the locus. In some embodiments, the observation is determined using the sequences generated in step 205. A locus (plural loci) may be a fixed position on a chromosome, like the position of a gene or a marker (genetic marker). The observation may be a variant of similar sequences located at a given locus, which is known as an allele. In some embodiments, a variant is a SNP. In other embodiments, a variant is a large structural variant such as a microdeletion or aneuploidy. At step 215, statistical association is performed for each locus of the genome having an observation using only the samples collected in step 210 that have the observation. At step 220, the statistical association performed for each site are combined using standard approaches for combining summary information from different GWAS performed on different populations, that is, correct for population structure and other confounding variants. Advantageously, since every site will be covered by a large set of samples (although a different set of samples for each locus), it is now possible to collect high-quality summary statistics for less-common variants, in a cost-effective manner.

In alternative embodiments, off-target lcWGS reads obtained from a sample are used for associating one or more microbiotic species or taxa with traits, lifestyle, or disease. For example, bacteria, viral, or mitochondrial nucleic acid obtained from a patient sample (off-target nucleic acid or reads that are not of the patients nucleic acid) may be used in the example process 200 for a low-coverage assay with a focus on less common variants (MAF<1%) as described with respect to FIG. 2 to associate one or more microbiotic species, viruses, or mitochondrial features with traits, lifestyle, or disease of the patient.

### Imputation for Low Coverage Sequencing

Technological advances have made genomewide association studies possible. Rather than genotyping <10,000 variants, these studies typically genotype 500,000-2,000,000 loci in the genome. However, for some applications (such as ancestry, or genome-wide-association-studies), the estimated genotypes in a larger set of an additional several million loci is important (e.g., >10 million common genetic variants are likely to exist). While in traditional genetic linkage and founder haplotype mapping studies, geneticists expect to identify long stretches of shared chromosome inherited from a relatively recent common ancestor, in genome wide association studies that focus on apparently unrelated individuals, geneticists expect to identify only relatively short stretches of shared chromosome. Remarkably, genotype imputation can use these short stretches of shared haplotype to estimate the effects of many variants that are not directly genotyped with great precision.

Genetic imputation is the process of using an individual's partial genotype to estimate other, unobserved genotypes from the same individual. Imputation is a statistical inference problem taking as input an individual's partial genotype as well as full genotype information for a large “reference population”. The reference population, or reference panel, is used to build the expected relationships between the genotypes in different loci in the genome, and these relationships are then applied to the partial genotype of the individual. As a very simple example, if 90% of the samples in the reference population that had the nucleotide A in position chr1:1000 in the genome also had the nucleotide C in position chr1:1100, and if we know that an individual has the nucleotide A in position chr1:1000 but we don't know the genotype or nucleotide in position chr1:1100, this unknown data may be imputed to be C with a 90% probability.

The relation between the size of the reference population and imputation quality is well-established, with larger populations producing more accurate imputation (that is, the estimated genotypes match the actual ones at higher rates). However, a larger reference population also means increased computation times for the imputation process, and in some instances with a quadratic relation: imputation using a reference of 1000 individuals requires 100 times more computational power than imputation using a reference of 100 individuals.

To overcome the challenges of using larger reference populations for imputation, various embodiments are directed to a two-tier assay: a fixed, small part of the genome (several million loci) may be consistently genotyped at high accuracy. In addition, a random large subset of the genome may be genotyped at decreased accuracy. The remainder of the genome is not observed directly, and requires imputing for applications such as those mentioned earlier. With this two-tier approach it is possible to utilize a mechanism that limits the amount of computation required, but still imputes the unobserved part of the genome with high accuracy.

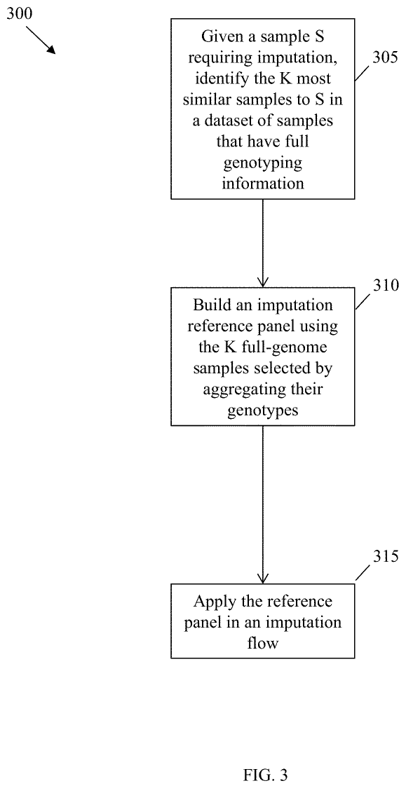

FIG. 3 is a block diagram that illustrates an example process 300 for imputation in a low-coverage assay, according to some embodiments. In step 305, given a sample S requiring imputation (e.g., a sample processed with low coverage whole genome sequencing as described in FIG. 1), identify the K most similar samples to S in a dataset of samples that have full genotyping information. In some embodiments, the dataset of samples is obtained from a database such as the 1000 genomes project, the HapMap Consortium database or a proprietary database. In certain embodiments, the data set of samples is a proprietary data set of WGS samples stored in a proprietary database. For computing similarity between samples, any number of well-established approaches can be used, for example Identity-by-Descent estimation or principal component analysis. At step 310, build an imputation reference panel using the K full-genome samples selected by aggregating their genotypes. At step 315, apply the reference panel in an imputation flow. In some embodiments, the flow may include identifying stretches of shared haplotype in the K full-genome samples and missing genotypes for each patient sample can be filled in by copying alleles observed in matching reference haplotypes. For example, when there is ambiguity about which haplotype stretch should be “copied” to fill in missing genotypes for a particular individual, imputation programs typically provide an answer that summarizes this ambiguity (for example, in 60% of reconstructions genotype A/C was observed at a specific site, whereas in the remaining 40% a different genotype A/A was observed). In principle, any of the methods typically used to estimate missing haplotypes—whether based on a simple heuristic or on an E-M algorithm or on more sophisticated coalescent models could be used to impute missing genotypes. Advantageously, evaluation of this approach shows performance that approaches that of using a much larger reference population, but at a fraction of the cost.

### Performing Analysis on Off-Target Reads

Targeted-panel next generation sequencing assays attempt to sequence a fixed subset of the genome by “targeting” only some regions in the whole genome. Chemical processes may be used to isolate the regions of interest from the rest of the genome before sequencing the nucleic acid, achieving higher overall cost effectiveness of the assay. However, these processes are imperfect, resulting in some amount of data being sequenced outside the regions being targeted; these are often referred to as “off-target reads”. Although the amount of these off-target reads is low compared to the reads targeted by the assay, their aggregate amount is typically sufficient to perform some of the tasks that a full low coverage whole genome sequencing panel would. These include: (i) Identifying the genetic ancestry of a sample, and consequently handling it differently in downstream processing; (ii) targeting specific areas for higher coverage, based on putative information about presence of genetic variants in these regions; and (iii) reducing or increasing targeted overall coverage in a subsequent low coverage whole genome sequencing assay, to achieve a minimum total number of “off-targeted” reads.

### Using a Combination of High Coverage Sequencing and Low Coverage Sequencing

In various embodiments, high coverage whole genome sequencing and low coverage whole genome sequencing may be used in conjunction for enhanced analysis. In some embodiments, initially low coverage whole genome sequencing is performed on a sample in accordance with processes discussed with respect to FIG. 1. Thereafter, analysis may be performed on the low coverage reads to identify regions or locations within the low coverage reads that are missing information (e.g., base pair identification) or that could use some increased detail. In some embodiments, these regions or locations may be the same locations identified during imputation analysis with unobserved genotypes. In addition to or alternative to performing imputation, high coverage sequencing may be performed on only those regions or locations that are determined to be missing information (e.g., base pair identification) or that could use some increased detail. The high coverage sequencing may be performed in a similar manner as described with respect to FIG. 1 but only on with those regions or locations that are determined to be missing information and with increased depth, e.g., >100×.

In other embodiments, initially high coverage whole genome sequencing is performed on a sample in a similar manner as described with respect to FIG. 1 but with increased depth, e.g., >100×. For example, high coverage sequencing may be used to determine ancestry of the patenting sample in order to select an appropriate reference panel for subsequent testing or analysis. Thereafter, low coverage sequencing may be performed as described with respect to FIG. 1 for further analysis such as in a GWAS. Essentially, the high coverage and low coverage sequencing may be implemented to balance accuracy and efficiency of the testing or analysis.

### Other Applications for Whole Genome Sequencing Data

In various embodiments, a method is provided for that transforms WGS data to find equivalency to microarray (non-next-gen-sequencing) data, and importantly to different specific microarray designs, so that the transformed data can be used in applications typically relying on array data. Microarrays representing collections of promoters, coding regions, transcript 3′ ends, alternative spliced exons, SNPs, and disease-gene arrays are all commonplace. However, microarray design requires a priori knowledge of the genome or genomic features. This directly affects array effectiveness in cases of incomplete, incorrect, or outdated genome annotations. In order to overcome the limitations of microarrays while still being able to use WGS data in applications typically relying on array data, some embodiments, provide for obtaining low-coverage WGS data, which is used to infer a genetic relationship between samples in a GWAS, and subsequently excluding closely related samples or otherwise correcting for bias closely related samples may introduce.

### Applying Low Coverage Whole Genome Sequencing for Intelligent Genomic Routing

In various embodiments, techniques are provided (e.g., a method, a system, non-transitory computer-readable medium storing code or instructions executable by one or more processors) for applying low coverage whole genome sequencing for intelligent genomic routing. Some embodiments are directed to a genomic routing system. The genomic routing system is configured to obtain genomic data and self-reported data for a subject (e.g., a patient or consumer), determine whether the genomic data and self-reported data of the subject satisfies eligibility criteria for a subset of genomic routes from a plurality of available genomic routes, and prioritize and select one or more genomic routes from the subset of genomic routes for the subject. As used herein “a genomic route” or “genomic routes” are pathways to insights and/or information supporting a product, service, event, or benefit for a subject that are differentiated based on underlying genomic data and self-reportable data for the subject.

Genomics offers opportunities for improving health without a thorough understanding of the underlying disease, disorder, condition, or syndrome. For example, conventionally a subject may seek out genomic direct-to-consumer services that include the subject providing a biological sample, a laboratory performing genomic analysis of the biological sample (e.g., biomarker identification, genotyping, ancestry analysis, risk of inheriting various diseases, etc.), and the laboratory directly reporting the results of the genomic testing to the subject. Many times the results of the genomic testing are provided with an explanation of potential risk factors and/or potential treatments without a thorough understanding of the underlying disease, disorder, condition, or syndrome (e.g., cancer therapies may be identified based on genomic profiles that identify tumor subtypes). A problem associated with these conventional genomic direct-to-consumer services is that the services typically focus on the genomic data (e.g., WGS data) and rarely contextualize the genomic data much less utilize information outside of the genomic data to provide a holistic health plan personal to the subject. Moreover, the results and information regarding the results provided by conventional genomic direct-to-consumer services are often misunderstood or unintentionally mislead the subjects seeking out the services, which ultimately results in underutilization of the results and information for directing change in the health of the subject.

To address these problems, various embodiments described herein are directed to genomic routing systems and methods capable of using genomic data to not only provide traditional information such as biomarker identification, genotyping, ancestry analysis, risk of inheriting various diseases, etc. but supplement that traditional information with a genomic route to insights and/or information supporting a product, service, event, or benefit personalized for the subject. For example, various embodiments of the present disclosure include a system including one or more processors and a memory coupled to the one or more processors. The memory is encoded with a set of instructions configured to perform a process including performing a lcWGS of a biological sample from a subject to obtain a set of reads, determining an inference of a phenotype from the set of reads, obtaining self-reported data from the subject, executing a first query on eligibility criteria for a plurality of genomic routes to obtain a set of genomic routes that satisfy the first query, executing a second query on routing criteria for the set of genomic routes to obtain a subset of ranked genomic routes that satisfy the second query, and selecting one or more genomic routes from the subset of ranked genomic routes based on the ranking of each of the one or more genomic routes. Advantageously, these techniques provide for a further deepening of our understanding of the underlying disease, disorder, condition, or syndrome and provide a holistic health plan personal to the subject that will accelerate the transition to genomic medicine (clinical care based on genomic information).

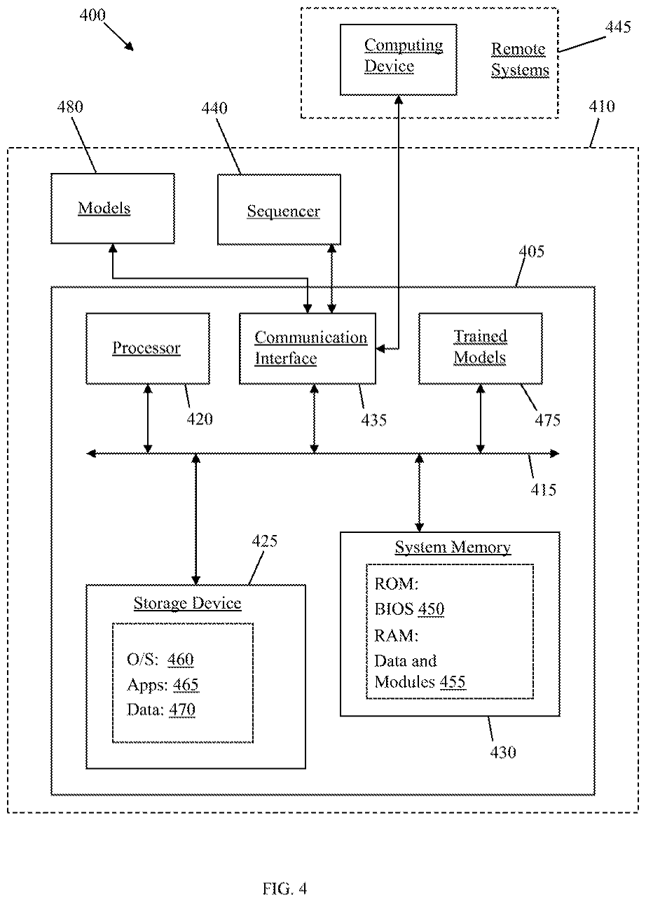

FIG. 4 is an illustrative architecture of a computing system 400 implemented in various embodiments. The computing system 400 is only one example of a suitable computing system and is not intended to suggest any limitation as to the scope of use or functionality of the various embodiments. Also, computing system 400 should not be interpreted as having any dependency or requirement relating to any one or combination of components illustrated in computing system 400.

As shown in FIG. 4, computing system 400 includes a computing device 405. The computing device 405 can be resident on a network infrastructure such as within a cloud environment, or may be a separate independent computing device (e.g., a computing device implemented within the environment of a medical laboratory 410 such as part of a genomic sequencing and analysis system). The computing device 405 may include a bus 415, processor 420, a storage device 425, a system memory (hardware device) 430, and a communication interface 435.

The bus 415 permits communication among the components of computing device 405. For example, bus 415 may be any of several types of bus structures including a memory bus or memory controller, a peripheral bus, and a local bus using any of a variety of bus architectures to provide one or more wired or wireless communication links or paths for transferring data and/or power to, from, or between various other components of computing device 405.

The processor 420 may be one or more integrated circuits, printed circuits, controllers, microprocessors, or specialized dedicated processors that include processing circuitry operative to interpret and execute computer readable program instructions, such as program instructions for controlling the operation and performance of one or more of the various other components of computing device 405 for implementing the functionality, steps, and/or performance of the embodiments discussed herein. In certain embodiments, processor 420 interprets and executes the processes, steps, functions, and/or operations, which may be operatively implemented by the computer readable program instructions. For example, processor 420 can obtain (e.g., WGS data) from a sequencer 440 and process/analyze the genomic data as described with respect to FIGS. 1-3, obtain and process self-reported data of the subject from one or more remote systems 445, and/or system memory 430, input two or more characteristics of the a genomic data and self-reported data into a query model to determine whether the genomic data and self-reported data of the subject satisfies eligibility criteria for at least a subset of genomic routes (one or more genomic routes) from a plurality of available genomic routes. When the genomic data and self-reported data of the subject satisfies eligibility criteria for at least a subset of genomic routes, input the two or more characteristics of the a genomic data and self-reported data into a ranking model to prioritize and select one or more genomic routes from the subset of genomic routes for the subject, and provide insights and/or information supporting a product, service, event, or benefit for a subject based on the selected one or more genomic routes. In some embodiments, the information obtained or generated by the processor 420, e.g., WGS data, self-reported data of the subject, etc., can be stored in the storage device 425.

The system memory 430 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system 450 (BIOS) including the basic routines that help to transfer information between the various other components of computing device 405, such as during start-up, may be stored in the ROM. Additionally, data and/or program modules 455 such as at least a portion of operating system 460, application programs 465, and/or program data 470, that are accessible to and/or presently being operated on by processor 420, may be contained in the system memory 430.

The data and/or program modules 455 may include a genomic data collector that is configured to generate, collect and/or save genomic data for each subject to the database (e.g., a database such as a table within the storage device 425). In some instances, the genomic data collector is configured to drive the sequencer 440, and push genomic data through the algorithms and models. The data and/or program modules 455 may further include a self-reported data collector configured to generate, collect, and/or maintain a comprehensive profile for each subject within the database (e.g., self-reported data associate with the genomic data and the subject in a table format). In some instances, the self-reported data collector is configured to obtain or collect the self-reported data directly from the subject (e.g., using a self-reporting form or template) and/or accesses one or more remote systems 445 (e.g., health care records or social media associated with the subject), and push the self-reported data through the algorithms and classifiers. The data and/or program modules 455 may further include a genomic routing module configured to generate and maintain genomic routes available to implement the routing features to provide insights and/or information supporting a product, service, event, or benefit for a subject. The data and/or program modules 455 may further include a controller module that includes an interface device driver for interfacing with other modules 455 and/or a user (e.g., an administrator), one or more models 475, 480 (e.g., a query dependent model, a decision tree, learn to rank model, etc.) configured to perform queries with evaluation, rank and prioritize genomic routes, and calculate or determine characteristics of the genomic data and the self-reporting data including (i) genotype and identification of genes for a particular disorder and/or risk factors for one or more diseases, disorders, conditions, and/or syndromes, (ii) presence and/or absence of biomarkers for a disease, (iii) identification of carrier status for recessively inherited disorders; (iv) subject profile (e.g., gender, ethnicity, age, etc.), (v) family medical history, (vi) environmental exposure (e.g., presence of drugs or chemicals in the subjects environment such as tobacco smoking or prolong exposure to asbestos, living environment including socio economic status, average temperature, and average sun exposure, etc.), and (vii) overall behavior analysis of the subject (e.g., identification of behavior that decreases or increase risk for gene mutations, behavior that increases or decreases risk for disease such as exercise or healthy eating, behavior associated with mental disease, etc.).

The communication interface 435 may include any transceiver-like mechanism (e.g., a network interface, a network adapter, a modem, or combinations thereof) that enables computing device 405 to communicate with remote devices or systems, such as medical laboratory 410, sequencer 440, a mobile device or other computing devices within remote systems 445 such as, for example, a server in a networked environment, e.g., cloud environment. For example, computing device 405 may be connected to remote devices or systems via one or more local area networks (LAN) and/or one or more wide area networks (WAN) using communication interface 435.

As discussed herein, computing system 400 may be configured to obtain genomic data and self-reported data for a subject (e.g., a patient or consumer), determine whether the genomic data and self-reported data of the subject satisfies eligibility criteria for a subset of genomic routes from a plurality of available genomic routes, and prioritize and select one or more genomic routes from the subset of genomic routes for the subject based on the underlying genomic data and self-reportable data for the subject. In particular, computing device 405 may perform tasks (e.g., process, steps, methods and/or functionality) in response to processor 420 executing program instructions contained in non-transitory machine readable storage medium, such as system memory 430. The program instructions may be read into system memory 430 from another computer readable medium (e.g., non-transitory machine readable storage medium), such as data storage device 425, or from another device via the communication interface 435 or server within or outside of a cloud environment. In some embodiments, hardwired circuitry of computing system 400 may be used in place of or in combination with the program instructions to implement the tasks, e.g., steps, methods and/or functionality, consistent with the different aspects discussed herein. Thus, the steps, methods and/or functionality disclosed herein can be implemented in any combination of hardware circuitry and software.

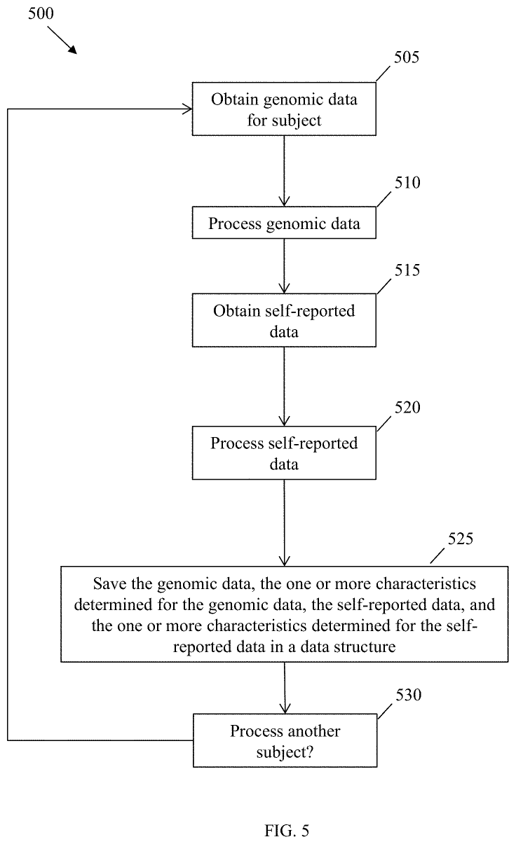

FIGS. 5, 6, and 7 are simplified flowcharts depicting processing performed for preparing or pre-processing genomic data and self-reported data for a subject (e.g., a patient or consumer), determining whether the genomic data and self-reported data of the subject satisfies eligibility criteria for a subset of genomic routes from a plurality of available genomic routes, prioritizing and selecting one or more genomic routes from the subset of genomic routes for the subject, and providing insights and/or information supporting a product, service, event, or benefit for a subject based on the selected one or more genomic routes according to various embodiments. The steps of FIGS. 5, 6, and 7 may be implemented in the system environment of FIG. 4, for example. As noted herein, the flowcharts of FIGS. 5, 6, and 7 illustrate the architecture, functionality, and operation of possible implementations of systems, methods, and computer program products according to various embodiments. In this regard, each block in the flowchart or block diagrams may represent a module, segment, or portion of code, which includes one or more executable instructions for implementing the specified logical functions. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combination of blocks in the block diagrams and/or flowchart illustration, can be implemented by special purpose hardware-based systems that perform the specified functions or acts, or combinations of special purpose hardware and computer instructions.

FIG. 5 depicts a simplified flowchart 500 illustrating a process used to prepare or pre-process genomic data (e.g., WGS or sequencing data obtained and described with respect to FIGS. 1-4) and self-reported data for a subject. At step 505, the genomic data is obtained for the subject using one or more processes 100, 200, and 300, as described with respect to FIGS. 1, 2, and 3, respectively. In some instances, the genomic data is low coverage whole genome sequencing data. In other embodiments, the genomic data is a combination of high coverage whole genome sequencing and low coverage whole genome sequencing. At step 510, the genomic data is processed to determine one or more characteristics of the genomic data. The processing of the genomic data may include pre-processing of the data and/or processing of the data using one or more tools. For example, the genomic data may be pre-processed to call bases, correct errors, determine depth coverage, perform quality control, count k-mers, etc. The genomic data may be processed to align reads, remove duplicates, calibrate or re-calibrate variants, align reads, evaluate alignments, etc. Thereafter, the processing may further include analyzing the pre-processed and/or processed data to detect single nucleotide variants (e.g., SNPs), detect indels, detect biomarkers, detect insertions/deletions, detect copy number variants, detect inversions, detect repeats, identify a genotype or phenotype, detect microsatellites, detect mutational signatures, analyze aneuploidy, infer sex, detect plasmids, analyze mitochondrial DNA, etc. In response to the processing, one or more characteristics of the genomic data are determined. In some instances, the characteristics include one or more of the following: (i) genotype and identification of genes for a particular disorder, (ii) risk score or factors for one or more diseases, disorders, conditions, and/or syndromes, (ii) presence and/or absence of biomarkers for a disease, (iii) single nucleotide variants, insertions/deletions, copy number changes, and large structural variants, (iv) identification of carrier status for recessively inherited disorders, and (v) identification of one or more microbiotic species or taxa associated with traits, lifestyle, or disease of the subject.

At step 515, the self-reported data is obtained for the subject. The self-reported data may be obtained by asking questions of the subject (e.g., providing the subject with self-reporting form or template such as a survey or questionnaire) and/or accessing and mining data from systems associated with the subject. In some instances, the questions and data mining may be implemented as part of a profile or account set-up process. For example, a subject may set-up a profile or account that includes completing a survey or questionnaire. The survey or questionnaire may include asking for biographical data and/or medical records such as name, gender, address, prior-addresses, employment, education level, clinical data, medical history, family medical history, demographics, vital signs, diagnoses, medications, treatment plans, progress notes, problems, immunization dates, allergies, radiology images, laboratory and test results, etc. The survey or questionnaire may further include asking for information from and/or access to remote systems that are associated with the subject such as social media accounts, health insurance accounts, medical record or charts, employment accounts, exercise monitoring accounts, etc.

At step 520, the self-reported data is processed to determine one or more characteristics of the self-reported data. The processing of the self-reported data may include pre-processing of the data and/or processing of the data using one or more tools. For example, the self-reported data may be pre-processed to remove special characters, remove punctuation, clean numbers, remove misspells, remove contractions, image transformations such as cropping, filtering, rotating or flipping images, etc. The self-reported data may be processed to query, normalize, organize such as create vectors or clusters for data, classify, etc. Thereafter, the processing may further include analyzing the pre-processed and/or processed data to determine a subject profile, determine a subject medical history, determine a family medical history, identify or determine environmental exposures (e.g., presence of drugs or chemicals in the subjects environment such as tobacco smoking or prolong exposure to asbestos, living environment including socio economic status, average temperature, and average sun exposure, etc.), identify behavior that decreases or increase risk for gene mutations, identify behavior that increases or decreases risk for disease such as exercise, healthy eating habits, or poor eating habits, identify behavior associated with mental disease, etc. In response to the processing, one or more characteristics of the self-reported data are determined. In some instances, the characteristics include one or more of the following: (i) gender, (ii) ethnicity, (iii) age, (iv) presence of a drug or chemical in the subjects environment, (v) a subject's medical treatment, (v) a subject's disease, disorder, condition, and/or syndrome diagnoses, (vii) a record from a subject's health care provider visit (e.g., a record of a mammogram or laboratory and test results), (viii) a behavior of the subject, (ix) a medical treatment of a relative of the subject, (x) disease, disorder, condition, and/or syndrome diagnoses for a relative of the subject, (xi) an exercise profile for the subject, and (xii) a eating profile for the subject.

At step 525, the genomic data, the one or more characteristics determined for the genomic data, the self-reported data, and the one or more characteristics determined for the self-reported data are saved in a data structure that may be queried and subject to one or more create, read, update or delete (CRUD) operations. For example, the genomic data, the one or more characteristics determined for the genomic data, the self-reported data, and the one or more characteristics determined for the self-reported data may be saved in a database table implemented in a storage device. The genomic data may be saved in the data structure in association with the one or more characteristics determined for the genomic data, the self-reported data, and the one or more characteristics determined for the self-reported data. At step 530, the process may be repeated for each subject to be added to the system.

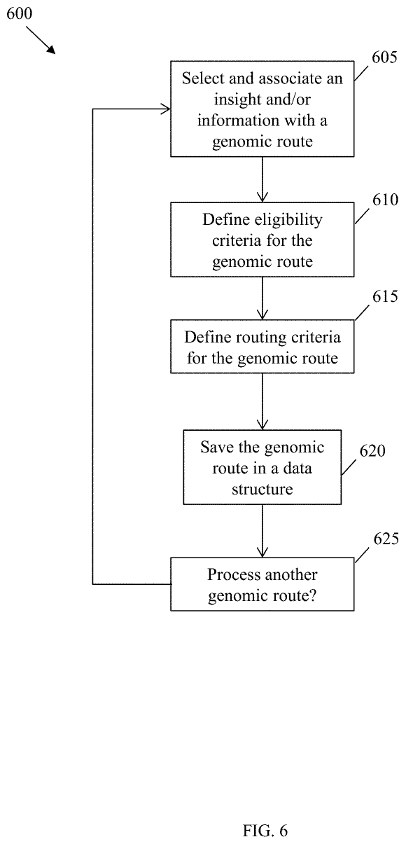

FIG. 6 depicts a simplified flowchart 600 illustrating a process used to generate one or more genomic routes. At step 605, an insight and/or information supporting a product, service, event, or benefit for a subject is selected and associated with a genomic route. In some instances, the insight and/or information pertains to a product, service, event, or benefit that a service provider (e.g., a genomic direct-to-consumer service provider) intends to provide to one or more subjects is defined for a genomic route. In other instances, the insight and/or information pertains to a product, service, event, or benefit that a third party service provider (e.g., a partner of the genomic direct-to-consumer service provider, a healthcare provider, or an insurance company) intends to provide to one or more subjects. For example, a genomic direct-to-consumer service provider may be interested in providing information supporting a gym membership at a partner company to a subject that is obese and at risk for heart disease. Alternatively, a third party service provider may be interested in providing a coupon for money off a next mammogram for subj ects that have a record of no mammogram in the last three years and are at risk for breast cancer. Alternatively, a third party service provider may be interested in providing an advertisement for an electric toothbrush for subjects that have a gene for greater risk of cavities and have a medical history of cavities. Alternatively, a genomic direct-to-consumer service provider may be interested in providing information supporting a mental check-up or psychiatric evaluation at a partner company qualified under the subject's health care insurance. In some instances, selecting the insight and/or information supporting a product, service, event, or benefit may include partnering with one or more third part service providers and/or soliciting offers from one or more third part service providers, maintaining a comprehensive list of potential insights and/or information supporting a product, service, event, or benefit (in some instances the list may include expiration dates for the various insights and/or information), and selecting an insight and/or information supporting a product, service, event, or benefit for a subject from the comprehensive list.

At step 610, eligibility criteria is defined for the genomic route. Eligibility criteria are query terms or conditions that may be queried and need to be satisfied for the genomic route to be consider a potential genomic route for a particular subject. The eligibility criteria are used to gate whether or not a genomic route should be considered for a subject, and consequently saves on computation power and increases robustness of the overall query and ranking process for selecting insights and/or information supporting a product, service, event, or benefit for a subject. In some instances, the eligibility criteria includes two or more conditions, for example between two and ten conditions that must be satisfied for the genomic route to be consider a potential genomic route for a particular subject. In other instances, the eligibility criteria includes two or more conditions, for example between two and twenty conditions and a certain percentage of those conditions (e.g., >70%) must be satisfied for the genomic route to be consider a potential genomic route for a particular subject. In various embodiments, the eligibility criteria or conditions for the eligibility criteria are defined based on the insight and/or information supporting a product, service, event, or benefit defined for the genomic route and one or more characteristics of the genomic data and/or the self-reported data that could be available and indicate a health condition that would benefit from the insight and/or information. For example, if the insight and/or information supports a gym membership (e.g., money off a gym local to the subject), then the eligibility criteria may include one or more characteristics of the genomic data and/or the self-reported data that indicate a health condition that would benefit from a gym membership and in some instances be accessible to the subject such as a risk factor or gene associated with obesity or heart disease and/or an age greater than eighteen.

At step 615, routing criteria is defined for the genomic route. Routing criteria are query terms or conditions that may be queried and are used by a ranking model to rank or prioritize the genomic routes for a subject. In some instances, the routing criteria includes two or more conditions, for example between two and a hundred conditions or two and a thousand conditions. In various embodiments, the routing criteria or conditions for the routing criteria are defined based on the insight and/or information supporting a product, service, event, or benefit defined for the genomic route and one or more characteristics of the genomic data and/or the self-reported data that could be available and indicate a health condition that would benefit from the insight and/or information. For example, if the insight and/or information supports a gym membership (e.g., money off a gym local to the subject), then the routing criteria may include one or more characteristics of the genomic data and/or the self-reported data that indicate a health condition that would benefit from a gym membership and in some instances be accessible to the subject such as a risk factor or gene associated with obesity or heart disease, a body fat index greater than 35%, a record of a heart attack, a record that indicates the subject is not currently exercising, a record that indicates the subject is not eating healthy foods, a record that indicates the subject's health insurance provides a discount on health insurance if they are enrolled and participate in a qualified exercise program, and/or an age greater than eighteen.

In various embodiments, defining the routing criteria includes indexing and weighting the routing criteria. Weighting is a process to assign a value to each term (e.g., each routing criteria) as it relates to the genomic route. The weighting is the assignment of numerical values to routing criteria that represent their importance to the genomic route in order to improve query and retrieval effectiveness. In some instances, the weighting considers the relative importance of the one or more characteristics of the genomic data and/or the self-reported data to the genomic route and overall health condition that would benefit from the insight and/or information, which can improve system effectiveness, since not all genomic routes in a genomic route collection are of equal importance to every health condition. Weighing the terms is the means that enables the retrieval system to determine the importance of a given characteristics of the genomic data and/or the self-reported data in a certain genomic route. In some embodiments, the one or more characteristics associated with the routing criteria include primary and secondary characteristics and thus carry a range of importance for a given genomic route and result in assignment of varied weights to the routing criteria for the genomic route.

For example, genomic data and/or self-reported data that includes a risk factor or gene associated with obesity or heart disease, a body fat index greater than 35%, a record of a heart attack may be classified as a primary characteristics for a genomic route relating to heart disease and receive a higher weight for such a genomic route. The risk factor or gene associated with obesity or heart disease and record of a heart attack may receive a higher weight as compared to the body fat index greater than 35% denoting a higher relevance for the risk factor or gene associated with obesity or heart disease and record of a heart attack in such a genomic route. Whereas a record that indicates the subject is not currently exercising, a record that indicates the subject is not eating healthy foods, a record that indicates the subject's health insurance provides a discount on health insurance if they are enrolled and participate in a qualified exercise program, and/or an age greater than eighteen may be classified as a secondary characteristics for a genomic route relating to heart disease and may receive a lower weight for such a genomic route. The secondary characteristics may receive lower weights than the primary characteristics for a given genomic route. Whereas genomic data that includes the presence of gene biomarkers BRCA1 or BRCA2 and/or self-reported data that includes a record of the presence of one or more of cancer antigen 15-3 (CA 15-3), cancer antigen 27.29 (CA 27.29), and carcinoembryonic antigen (CEA) may not be considered relevant at all for a genomic route pertaining to heart disease and thus may not be labeled as primary or second characteristics for the genomic route and receive a weight of zero.

At step 620, the genomic route is saved in a data structure that may be queried and subject to one or more create, read, update or delete (CRUD) operations. For example, the genomic route may be save in a database table implemented in a storage device. The genomic route may be saved in the data structure in association with insight and/or information supporting a product, service, event, or benefit selected for the genomic route and the eligibility criteria and routing criteria defined for the genomic route. At step 625, the process may be repeated for each genomic route to be added to the system.

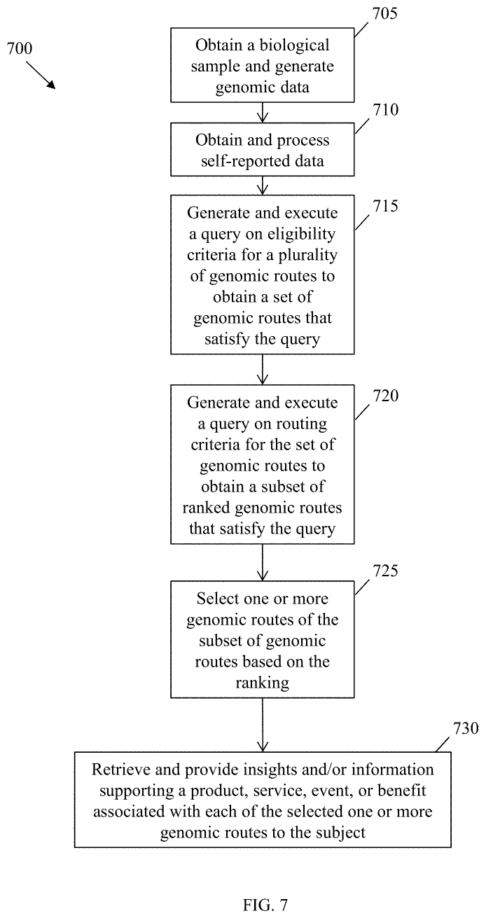

FIG. 7 depicts a simplified flowchart 700 illustrating a process used to query, rank, and select one or more genomic routes for a subject. At step 705, a biological sample is obtained from a subject and processed to generate genomic data (e.g., WGS data) and determine one or more characteristics for the genomic data. The genomic data may be obtained and processed as described with respect to FIGS. 1, 2, 3, and 5. At step 710, self-reported data is obtained for the subject and processed to determine one or more characteristics for the self-reported data. The self-reported data may be obtained and processes as described with respect to FIG. 5.

At step 715, a query is generated and executed on eligibility criteria for a plurality of genomic routes to obtain a set of genomic routes that satisfy the query. The query is generated using one or more query terms. The query terms include one or more characteristics for the genomic data and optionally one or more characteristics for the self-reported data. For example, the query may be generated to comprise a risk score or factor for one or more diseases, disorders, conditions, and/or syndromes determined via a lcWGS. In some embodiments, the query is executed in a query model. A query model such as query dependent model (e.g., a Boolean model) may be configured to retrieve a set of genomic routes from the plurality of genomic routes based on occurrences of the query terms in the eligibility criteria for each genomic route within the set of genomic routes. The query model may predict whether each genomic route is relevant to the query or not, but may not predict a degree of relevance for each genomic route.

At step 720, a query is generated and executed on routing criteria for the set of genomic routes to obtain a ranked subset of genomic routes that satisfy the query. The query is generated using one or more query terms. The query terms include one or more characteristics for the genomic data and one or more characteristics for the self-reported data. For example, the query may be generated to comprise a risk score or factor for one or more diseases, disorders, conditions, and/or syndromes determined via a lcWGS and a medical record retrieved from self-reported data. In some embodiments, the query is executed in a query model. A query and rank model such as learning to rank model (e.g., a Ranking SVM, IR SVM, AdaRank, LambdaRank, and LambdaMART models) may be trained retrieve a subset of genomic routes from the set of genomic routes based on occurrences of the query terms in the routing criteria for each genomic route. The query and rank model may predict whether each genomic route is relevant to the query or not, and predict a degree of relevance for each genomic route (rank each genomic route in relation to other genomic routes). As such, the query and rank model may be trained to rank or prioritize each genomic route that satisfies the query.

In various embodiments, the query and rank model is a learning to rank model configured to rank the rank or prioritize each genomic route that satisfies the query. The learning to rank algorithm may learn to directly rank items by training a query and rank model to predict the probability of a certain genomic route ranking over another genomic route. This may be done by learning a scoring function where genomic routes ranked higher should have higher scores. The query and rank model may be trained via gradient descent on a loss function defined over these scores. For each genomic route, gradient descent pushes the score up for every genomic route that ranks below it and pushes the score down for every genomic route that ranks above it. The “strength” of the push is determined by the difference in scores. To ensure that the query and rank model focuses on getting the higher ranks (which are generally more important) correct, weights may be applied to the “strength” of the push by a factor that accounts for how important the ranking is and the weights defined for the routing criteria.

At step 725, one or more genomic routes are selected from the subset of genomic routes for the subject. The one or more genomic routes are pathways to insights and/or information supporting a product, service, event, or benefit for a subject that are differentiated based on the underlying genomic data and self-reportable data for the subject. In various embodiments, the one or more genomic routes are selected based on the ranking of each of the genomic routes within the subset of genomic routes. In some embodiments, only the top ranking genomic route is selected. In other embodiments, a predetermined number (e.g., five) of the top ranking genomic routes are selected. At step 730, the insights and/or information supporting a product, service, event, or benefit associated with each of the selected one or more genomic routes are retrieved and provided to the subject, e.g., as an offering in response to the subject requesting sequencing analysis of their biological sample. In some embodiments, the insights and/or information supporting a product, service, event, or benefit are provided to the subject without the genomic data (e.g., the results of the sequencing analysis of their biological sample). In other embodiments, the insights and/or information supporting a product, service, event, or benefit are provided to the subject with the genomic data (e.g., the results of the sequencing analysis of their biological sample).

### Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A method, comprising:
performing, at a processing system, a low coverage whole genome sequencing of a biological sample from a subject to obtain a set of reads;
evaluating, at a processing system, coverage of the set of reads;
performing, by the processing system, statistical association for loci of the set of reads having an observation;
determining, by the processing system, an inference of a phenotype from the set of reads based on the statistical association;
obtaining, by the processing system, self-reported data from the subject;
executing, by the processing system, a first query on eligibility criteria for a plurality of genomic routes to obtain a set of genomic routes that satisfy the first query, wherein the first query includes the phenotype;
executing, by the processing system, a second query on routing criteria for the set of genomic routes to obtain a subset of ranked genomic routes that satisfy the second query, wherein the second query includes the phenotype and at least one piece of information from the self-reported data; and
selecting, by the processing system, one or more genomic routes from the subset of ranked genomic routes based on the ranking of each of the one or more genomic routes.

2. The method of claim 1, further comprising providing, by the processing system, insight and/or information supporting a product, service, event, or benefit associated with each of the selected one or more genomic routes to the subject.

3. The method of claim 1, the first query is executed on a query dependent model configured to obtain each genomic route of the plurality of genomic routes that satisfies the first query, and the second query is executed on a learning to rank model trained to rank or prioritize each genomic route of the set of genomic routes that satisfies the query.

4. The method of claim 1, further comprising:
identifying, by the processing system, within the set of reads one or more off-target reads;
performing, by the processing system, a statistical association for loci of the one or more off-target reads having an observation; and
determining, by the processing system, an inference of another phenotype from the one or more off-target reads based on the statistical association,
wherein the second query includes the phenotype, the another phenotype, and at least one piece of information from the self-reported data.

5. The method of claim 1, further comprising:
performing, at the processing system, a high coverage whole genome sequencing of the sample to obtain another set of reads; and
performing, by the processing system, statistical association for loci of the another set of reads having an observation,
wherein the inference of the phenotype is determined from the set of reads and the another set of reads based on the statistical association of both the set of reads and the another set of reads.

6. The method of claim 2, wherein the insight and/or the information are provided to the subject without the phenotype.

7. The method of claim 2, wherein the insight and/or the information are provided to the subject with the phenotype.

8. A system comprising:
one or more processors; and
memory coupled to the one or more processors, the memory encoded with a set of instructions configured to perform a process comprising:
performing a low coverage whole genome sequencing of a biological sample from a subject to obtain a set of reads;
evaluating coverage of the set of reads;
performing statistical association for loci of the set of reads having an observation;
determining an inference of a phenotype from the set of reads based on the statistical association;
obtaining self-reported data from the subject;
executing a first query on eligibility criteria for a plurality of genomic routes to obtain a set of genomic routes that satisfy the first query, wherein the first query includes the phenotype;
executing a second query on routing criteria for the set of genomic routes to obtain a subset of ranked genomic routes that satisfy the second query, wherein the second query includes the phenotype and at least one piece of information from the self-reported data; and
selecting one or more genomic routes from the subset of ranked genomic routes based on the ranking of each of the one or more genomic routes.

9. The system of claim 8, wherein the process further comprises providing insight and/or information supporting a product, service, event, or benefit associated with each of the selected one or more genomic routes to the subject.

10. The system of claim 8, the first query is executed on a query dependent model configured to obtain each genomic route of the plurality of genomic routes that satisfies the first query, and the second query is executed on a learning to rank model trained to rank or prioritize each genomic route of the set of genomic routes that satisfies the query.

11. The system of claim 8, wherein the process further comprises:
identifying within the set of reads one or more off-target reads;
performing a statistical association for loci of the one or more off-target reads having an observation; and
determining an inference of another phenotype from the one or more off-target reads based on the statistical association,
wherein the second query includes the phenotype, the another phenotype, and at least one piece of information from the self-reported data.

12. The system of claim 8, wherein the process further comprises:
performing a high coverage whole genome sequencing of the sample to obtain another set of reads; and
performing statistical association for loci of the another set of reads having an observation,
wherein the inference of the phenotype is determined from the set of reads and the another set of reads based on the statistical association of both the set of reads and the another set of reads.

13. The system of claim 9, wherein the insight and/or the information are provided to the subject without the phenotype.

14. The system of claim 9, wherein the insight and/or the information are provided to the subject with the phenotype.

15. A non-transitory computer readable storage medium storing instructions that, when executed by one or more processors of a computing system, cause the computing system to perform a process comprising:
performing a low coverage whole genome sequencing of a biological sample from a subject to obtain a set of reads;
evaluating coverage of the set of reads;
performing statistical association for loci of the set of reads having an observation;
determining an inference of a phenotype from the set of reads based on the statistical association;
obtaining self-reported data from the subject;
executing a first query on eligibility criteria for a plurality of genomic routes to obtain a set of genomic routes that satisfy the first query, wherein the first query includes the phenotype;
executing a second query on routing criteria for the set of genomic routes to obtain a subset of ranked genomic routes that satisfy the second query, wherein the second query includes the phenotype and at least one piece of information from the self-reported data; and
selecting one or more genomic routes from the subset of ranked genomic routes based on the ranking of each of the one or more genomic routes.

16. The non-transitory computer readable storage medium of claim 15, wherein the process further comprises providing insight and/or information supporting a product, service, event, or benefit associated with each of the selected one or more genomic routes to the subject.

17. The non-transitory computer readable storage medium of claim 15, the first query is executed on a query dependent model configured to obtain each genomic route of the plurality of genomic routes that satisfies the first query, and the second query is executed on a learning to rank model trained to rank or prioritize each genomic route of the set of genomic routes that satisfies the query.

18. The non-transitory computer readable storage medium of claim 15, wherein the process further comprises:
identifying within the set of reads one or more off-target reads;
performing a statistical association for loci of the one or more off-target reads having an observation; and
determining an inference of another phenotype from the one or more off-target reads based on the statistical association,
wherein the second query includes the phenotype, the another phenotype, and at least one piece of information from the self-reported data.

19. The non-transitory computer readable storage medium of claim 15, wherein the process further comprises:
performing a high coverage whole genome sequencing of the sample to obtain another set of reads; and
performing statistical association for loci of the another set of reads having an observation,
wherein the inference of the phenotype is determined from the set of reads and the another set of reads based on the statistical association of both the set of reads and the another set of reads.

20. The non-transitory computer readable storage medium of claim 16, wherein the insight and/or the information are provided to the subject without the phenotype.
